(19) United States
(12) Patent Application Publication
(10) Pub. No.: US 2024/0323658 A1
(43) Pub. Date: Sep. 26, 2024

(54) MEDICATION COMPLIANCE MONITORING DEVICE, SYSTEM, AND RELATED TECHNIQUES

(71) Applicant: ProMed Innovations LLC, Alpharetta, GA (US)

(72) Inventor: John Thomas McGibbon, Edgewater, FL (US)

(21) Appl. No.: 18/612,249

(22) Filed: Mar. 21, 2024

Related U.S. Application Data
(60) Provisional application No. 63/454,189, filed on Mar. 23, 2023.

(51) Int. Cl. H04W 4/38; G06K 19/077; G16H 20/13; H04W 12/50

(52) U.S. Cl. CPC H04W 4/38; G06K 19/07749; G16H 20/13; H04W 12/50

(57) ABSTRACT

A wireless tag apparatus, system, and related medication compliance monitoring techniques are disclosed. In some embodiments, the apparatus may include a sensing circuit configured to scan a sensor array associated with a medication dispenser external to the apparatus to determine whether contents of at least one medication compartment of the medication dispenser have been expelled. The apparatus further may include a transmitter configured to transmit a signal including data pertaining to whether the contents of the at least one medication compartment have been expelled, wherein the signal is a radio frequency signal. The apparatus may be configured to perform such scanning and/or transmitting based on the expiry of a time interval, which may be pre-programmed. In some embodiments, transmission may depend on acceptance of a secure pairing request from a computing device in communication with the apparatus. In some embodiments, the apparatus may be configured for use, for example, in monitoring medication compliance.

### CROSS-REFERENCE TO RELATED APPLICATIONS

This patent application claims the benefit of U.S. Provisional Patent Application No. 63/454,189, titled “Medication Compliance Monitoring Device, System, and Related Techniques,” filed on Mar. 23, 2023, which is herein incorporated by reference in its entirety.

### FIELD OF THE DISCLOSURE

The present disclosure is generally related to medication compliance monitoring and, more specifically, to devices, systems, and techniques related to medication compliance monitoring.

### BACKGROUND

Medications used in health care and treatment are often prescribed by medical professionals with instructions for the patient to take specific doses of the medication at specific times or intervals. To assist with proper dosage, the medications are often packaged in compartments of a blister package (i.e., a plastic material container comprising a number of sealable compartments having an open side intended to accommodate a drug dosage). These compartments are covered with a breakable cover. The blister pack is covered with a single breakable material, such as foil, or individual breakable flaps, in order to obtain a tight closure of the compartments and secure individual packaging. Generally, the compartments are arranged as a matrix configuration including a number of lines and/or columns. Typically, the rows can be times of the day, and the columns can be days of the week or month.

Ensuring a patient takes the appropriate dose of medication at the instructed time or interval is often a key aspect to successful medical treatment. Some prior art devices provide for monitoring or detection of the blister pack to identify when medication is removed from the blister pack. However, these devices are unable to monitor the blister pack fully (e.g., they are unable to monitor each individual medication compartment, which medication is taken, at what time, etc.) to ensure that the proper medication is taken by the patient. Furthermore, these conventional systems are often cumbersome to use, as they require hardwired power supplies or battery-based power supplies which frequently require recharging, and they often require additional hardware for enabling any communication of the monitored condition of the blister pack, among other shortcomings.

Thus, a heretofore unaddressed need exists in the industry to address the aforementioned deficiencies and inadequacies.

### SUMMARY

One example embodiment provides a wireless tag apparatus. The wireless tag apparatus may include a timer configured to count a designated time interval. The wireless tag apparatus also may include a sensing circuit configured to scan, after the time interval has elapsed, a sensor array associated with a medication dispenser external to the wireless tag apparatus to detect a change to an electrical circuitry of the sensor array corresponding to expulsion of contents of at least one medication compartment of the medication dispenser, wherein the change to the electrical circuitry comprises a breakage of the electrical circuitry disposed over the at least one medication compartment. The wireless tag apparatus also may include non-volatile memory configured to store data pertaining to whether the electrical circuitry of the sensor array has so changed. The wireless tag apparatus also may include a transceiver configured to transmit, after the time interval has elapsed, a first radio frequency (RF) signal including the data pertaining to whether the electrical circuitry of the sensor array has so changed. The wireless tag apparatus may be configured to transmit, in the first RF signal, all scanned sensor array data stored to date when a secure pairing request from an external computing device is accepted by the wireless tag apparatus.

Another example embodiment provides a method of medication compliance monitoring. The method may include counting a designated time interval. The method also may include scanning, after the time interval has elapsed, a sensor array associated with a medication dispenser to detect a change to an electrical circuitry of the sensor array corresponding to expulsion of contents of at least one medication compartment of the medication dispenser, wherein the change to the electrical circuitry comprises a breakage of the electrical circuitry disposed over the at least one medication compartment. The method also may include storing data pertaining to whether the electrical circuitry of the sensor array has so changed. The method also may include transmitting, after the time interval has elapsed, a radio frequency (RF) signal including either: (a) the data pertaining to whether the electrical circuitry of the sensor array has so changed; or (b) all scanned sensor array data stored to date when a secure pairing request from an external computing device is accepted.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a schematic illustration of a system for wireless medication compliance sensing, in accordance with an embodiment of the present disclosure.

FIG. 2 is a side view schematic illustration of the system for wireless medication compliance sensing of FIG. 1, in accordance with an embodiment of the present disclosure.

FIG. 3 is a top view schematic illustration of the sensor array over an opening in the system for wireless medication compliance sensing of FIG. 1, in accordance with an embodiment of the present disclosure.

FIG. 4 is a schematic illustration of the sensor array over openings in the system for wireless medication compliance sensing of FIG. 1, in accordance with an embodiment of the present disclosure.

FIG. 5 is a schematic illustration of an equivalent circuit of the sensor array of FIG. 4, in accordance with an embodiment of the present disclosure.

FIG. 6 is a schematic illustration of the sensor array for a housing with a 3×7 array of medication compartment openings in the system for wireless medication compliance sensing of FIG. 1, in accordance with an embodiment of the present disclosure.

FIG. 7 is a schematic of the wireless tag of the system for wireless medication compliance sensing, in accordance with an embodiment of the present disclosure.

FIG. 8 is a schematic of the system for wireless medication compliance sensing, in accordance with an embodiment of the present disclosure.

FIG. 9 is a side-view schematic illustration of the connectable wireless tag for use with the system for wireless medication compliance sensing, in accordance with an embodiment of the present disclosure.

FIG. 10 is a top view schematic illustration of the connectable wireless tag for use with the system for wireless medication compliance sensing, in accordance with an embodiment of the present disclosure.

FIG. 11 is a flowchart of a method for wirelessly sensing medication compliance, in accordance with an embodiment of the present disclosure.

FIG. 12 illustrates a medication compliance monitoring system configured in accordance with an embodiment of the present disclosure.

FIG. 13 is a block diagram of a wireless tag apparatus configured in accordance with an embodiment of the present disclosure.

FIG. 14 is a diagram of a sensor array configured in accordance with an embodiment of the present disclosure.

FIG. 15 is a flow diagram illustrating a method of medication compliance monitoring in accordance with an embodiment of the present disclosure.

### DETAILED DESCRIPTION

A wireless tag apparatus, system, and related medication compliance monitoring techniques are disclosed. In some embodiments, the apparatus may include a sensing circuit configured to scan a sensor array associated with a medication dispenser external to the apparatus to determine whether contents of at least one medication compartment of the medication dispenser have been expelled. The apparatus further may include a transmitter configured to transmit a signal including data pertaining to whether the contents of the at least one medication compartment have been expelled, wherein the signal is a radio frequency signal. In some embodiments, the apparatus may be configured to perform such scanning and/or transmitting based on the expiry of a time interval, which may be pre-programmed. In some embodiments, transmission may depend on acceptance of a secure pairing request from a computing device in communication with the apparatus. In some embodiments, the apparatus may be configured for use, for example, in monitoring medication compliance.

### I—Example Device, System Architecture, and Operation

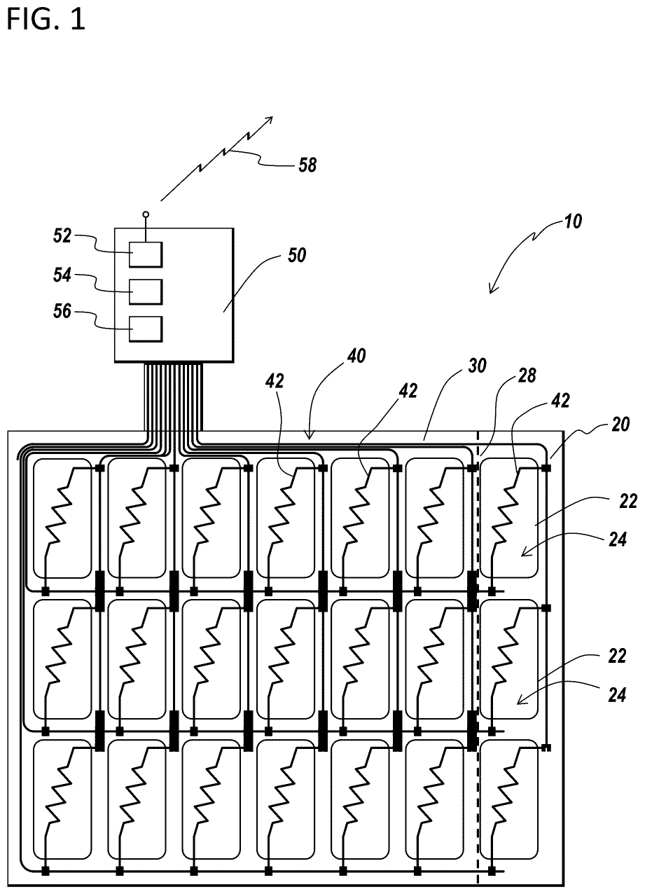

FIG. 1 is a schematic illustration of a system for wireless medication compliance sensing 10, in accordance with an embodiment of the present disclosure. The system for wireless medication compliance sensing 10 may include a housing 20 having a plurality of medication compartments 22. A breakable substrate 30 may be positioned on at least one side of housing 20, wherein breakable substrate 30 may cover an opening 24 to the plurality of medication compartments 22. A sensor array 40 may be positioned on the at least one side of housing 20. Sensor array 40 may have a plurality of resistors 42, wherein one of the plurality of resistors 42 is positioned across one of the openings 24 of the plurality of medication compartments 22.

A wireless tag 50 may be connectable to sensor array 40. Wireless tag 50 may have a transmitter 52, an accelerometer 54, and a sensing circuit 56, wherein sensing circuit 56 may be adapted to scan sensor array 40 upon activation of accelerometer 54, and wherein transmitter 52 may transmit a signal 58 having scanned sensor array data externally from wireless tag 50.

This system 10 may provide a convenient way to verify when medication is removed from a blister pack or similar medication housing, where the medication is stored in a particular arrangement for patient retrieval. The verification of medication compliance is one aspect of improving the health, care, and treatment of patients who must take a variety of medications periodically. Housing 20 may have a variety of sizes and may include any quantity of medication compartments 22 in a variety of patterns or arrays. For example, an array of 3×1, 5×1, 3×7, or 5×7 medication compartments 22 may be used, among other sizes. The quantity of medication compartments 22 may be keyed to the scheduled frequency of the patient taking the medication housed therein.

Although FIG. 1 does not illustrate the medication, it may be packaged within medication compartments 22 of housing 20, which together may be considered a blister pack, as is known in the art. Medication compartments 22 may be formed from molding or otherwise creating individual compartments within a single plastic sheet or a sheet made from another material. Each medication compartment 22 may have an opening 24, which may be sealed with breakable substrate 30. Breakable substrate 30 may be, for example, a paper-based substrate, a foil-based substrate, or another material capable of being broken or punctured by the user in a position over openings 24, thereby allowing the user to access medication held within medication compartment 22. Breakable substrate 30 may be positioned on housing 20, such as by being affixed to housing 20 with an adhesive or sealing technique, to seal the medication within medication compartments 22. When the medication is filled in each medication compartment 22 with breakable substrate 30 applied to housing 20, the medication may be sealed hermetically within medication compartments 22.

System 10 may use sensor array 40 and wireless tag 50 to monitor when medication has been removed from medication compartments 22. Specifically, sensor array 40 may include conductive lines positioned around medication compartments 22 with individual resistors 42 positioned over openings 24 of medication compartments 22. Sensor array 40 may be created by various techniques, such as by printing electrically conductive ink directly onto breakable substrate 30, by forming the conductive lines on another substrate which is positioned abutting or substantially abutting breakable substrate 30, or with other suitable methods, as will be apparent in light of this disclosure. The end result may be that sensor array 40 is formed with resistors 42 positioned over openings 24 such that, when breakable substrate 30 is punctured over openings 24, resistor 42 corresponding to that opening 24 is severed.

Wireless tag 50 may be connectable to sensor array 40, often removably connectable such that wireless tag 50 may be used repeatedly for subsequent blister packs. Wireless tag 50 may have a transmitter 52 for transmitting signals externally from wireless tag 50, an accelerometer 54 for monitoring a movement or motion of housing 20, such as when it is moved by the user, and a sensing circuit 56 including various circuitry for periodically scanning sensor array 40. For example, sensing circuit 56 may scan sensor array 40 upon activation of accelerometer 54 (e.g., upon movement of housing 20) to scan the operational state of each resistor 42 within sensor array 40, which in turn can be indicative of whether medication has been removed from a medication compartment 22 corresponding to that resistor 42. It may be common for sensing circuit 56 to scan sensor array 40 only upon activation of accelerometer 54 to preserve battery life of wireless tag 50 in comparison to constant or periodic scans of sensor array 40. In one of many alternatives, sensing circuit 56 may scan sensor array 40 at low-frequency, spaced intervals (e.g., one scan per 10 seconds) when accelerometer 54 is not activated and high-frequency, spaced intervals (e.g., 10 scans per second) when accelerometer 54 is activated.

Initially, sensing circuit 56 and/or processor 62 may detect when wireless tag 50 is first connected to sensor array 40, and it may scan sensor array 40 to determine if there is a good connection by testing each resistor 42 and the size of the matrix of sensor array 40. Sensing circuit 56 may sense the number of rows and columns within sensor array 40 to identify the size of sensor array 40. As an example, a 3×7 sensor array 40 may require in the range of ten to twenty-one contacts between wireless tag 50 and sensor array 40.

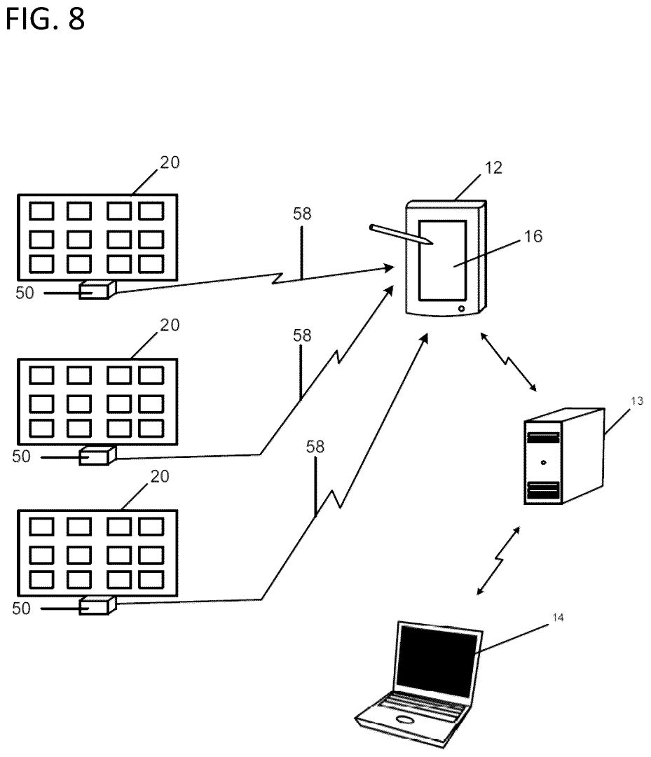

The scan may result in scanned sensor array data, which may include any data pertinent to sensor array 40 or wireless tag 50 itself. For example, the scanned sensor array data may include an identification of wireless tag 50, a functioning state of wireless tag 50, a state of each of the plurality of resistors 42 (e.g., whether each resistor 42 is severed or not), a size of sensor array 40, a size of the blister pack, or other information. Identification of wireless tag 50 also may be retrieved using a serial number chip stored on the sensor package. Signals communicated may be used to read the serial number. While transmission of the scanned sensor array data may not be required after each scan, transmitter 52 may transmit signal 58 externally from wireless tag 50 to communicate the scanned sensor array data to another device, such as a mobile electronic device, a server, or other database which stores or processes the scanned sensor array data, as discussed further relative to FIG. 8.

System 10 may include a number of additional features and components. For example, as is shown in FIG. 1, housing 20 may be perforated at perforation line 28 to allow for a portion of medication compartments 22 to be separated or removed from housing 20 as a whole. The ability to separate a portion of medication compartments 22 may allow a user to take a few days of medication instead of the entire housing 20 when they go on a weekend trip or other excursion where it may not be desirable to take the entire housing 20. Sensor array 40 still may be capable of operating after removal of the perforated portion of medication compartments 22.

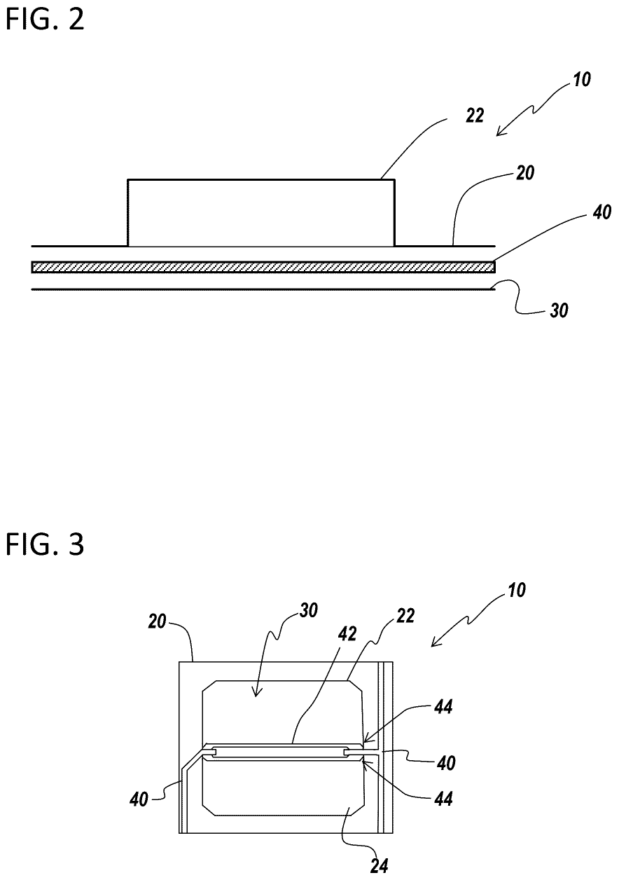

FIG. 2 is a side view schematic illustration of the system for wireless medication compliance sensing 10 of FIG. 1, in accordance with an embodiment of the present disclosure. Specifically, FIG. 2 illustrates the detailed layers of housing 20, breakable substrate 30, and sensor array 40 in one example. As is shown, housing 20 may form the top layer as a plastic bubble array having medication compartment 22 integrally formed within housing 20 material. Sensor array 40 may be positioned as a middle layer, proximate or abutting housing 20, and may be printed on a thin layer of material, such as a 2-mm or 3-mm layer of plastic. Breakable substrate 30 may be formed from a conventional foil-backed paper, or similar material, and positioned as the bottom layer. An adhesive, such as a pressure adhesive or thermal adhesive, may be applied to all surfaces to hermetically seal the three materials together.

FIG. 3 is a top view schematic illustration of sensor array 40 over an opening 24 in the system for wireless medication compliance sensing 10 of FIG. 1, in accordance with an embodiment of the present disclosure. As is shown, sensor array 40 may have a plurality of conductive traces which are positioned proximate to medication compartment 22 on housing 20. Resistor 42 of sensor array 40 may be electrically connected to the conductive traces of sensor array 40 and positioned over opening 24. A portion of resistor 42, for example, the crossbar, not the electrically conductive portion, may be cut at its ends 44 (e.g., a location along resistor 42 where it is positioned at an edge of opening 24) to allow resistor 42 to be severed easily. When breakable substrate 30 is printed on an insulating plastic material, the portion of breakable substrate 30 which contains opening 24 may be die-cut to allow the user to easily gain access to medication compartment 22, such as, for example, by punching or forcing the portion of breakable substrate 30 aligned with opening 24 until it severs along the die-cut outline (outline of opening 24). Ends 44 of resistor 42 preferably may be aligned with the die-cut outline to ensure resistor 42 is severed when the patient accesses medication compartment 22.

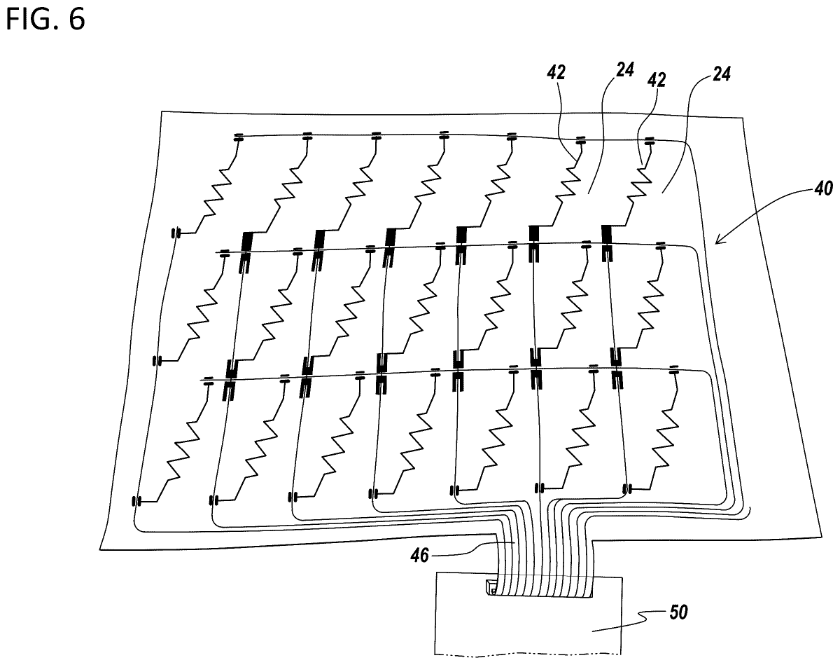

The manufacture of sensor array 40 on breakable substrate 30 may vary. For example, when sensor array 40 is printed on a plastic insulating material which is die-cut, a final layer of material may be required on the backside of the plastic insulating material to seal sensor array 40 therein. This final layer may be a non-conductive layer which ensures successful sensor array 40 operation. In another example, sensor array 40 may be printed on a paper-based breakable substrate 30 (as shown in FIG. 6), where no additional layers to seal sensor array 40 are required. In any manufacturing process of sensor array 40 on breakable substrate 30, it may be desirable to ensure that all layers of breakable substrate 30 may be punctured or ripped easily at the location along sensor array 40 to ensure circuit breakage.

Sensor array 40 with resistor 42, as shown in FIG. 3, may be manufactured with any of a wide range of suitable manufacturing processes, as will be apparent in light of this disclosure, thereby providing a system 10 with a sensor array 40 that can be tested fully before final assembly of system 10 with the medication. In one example of manufacturing sensor array 40, the conductors first may be printed with a highly conductive ink on breakable substrate 30. Then, insulating crossovers may be printed where the horizontal and vertical lines of sensor array 40 cross. Resistors and crossover connections (on the insulating crossovers) then may be printed with resistive ink or another non-conductive barrier. A further description of the manufacturing process of breakable substrate 30 and/or sensor array 40 is provided relative to FIG. 6.

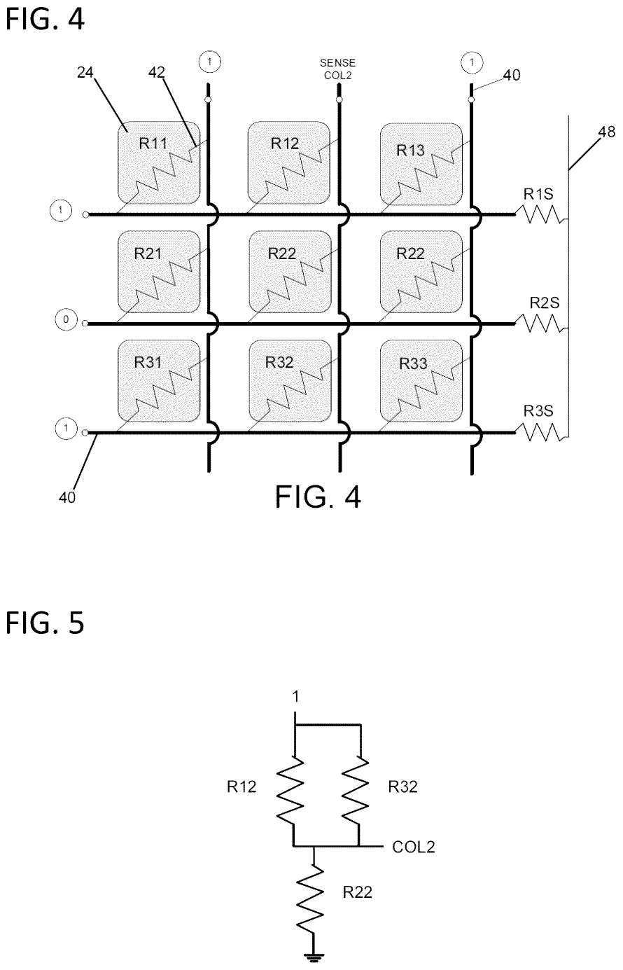

FIG. 4 is a schematic illustration of sensor array 40 over openings 24 in the system for wireless medication compliance sensing 10 of FIG. 1, in accordance with an embodiment of the present disclosure. As is shown in FIG. 4, sensor array 40 may be positioned proximate to each opening 24, and a resistor 42 may be positioned over each opening 24. Sensor array 40 may include conductive traces formed as columns and rows, each of which may be identified with a ‘1’ or ‘0’ as in FIG. 4. Similarly, each resistor 42 in FIG. 4 may be labeled (e.g., R11, R12, R13, etc.) as in FIG. 4. One example benefit of system 10 may be the ability to sense each opening 24 individually, which may be accomplished by scanning each resistor 42 independently of other resistors 42 within sensor array 40. Prior art techniques to sense sensor arrays, such as those used in computer keyboards, are incapable of sensing resistors 42 of this type on an individual level because only one to three keys can be closed simultaneously. In contrast, within system 10, all locations may be closed initially, and the sensing of each resistor 42 may be accomplished by opening each location as it needs to be detected. As an added benefit, this ability to sense locations individually may be done using very little power, thereby allowing the battery power of wireless tag 50 to run sensing circuit 56 for extended periods (e.g., a year or more) on a single coin cell battery. In some cases, the number of rows and verification of system connection(s) in the array may be determined with an extra column connection with a resistor connecting this column to each row.

It is also noted that system 10 may include a sensor array 40 with additional columns or rows of conductive traces. These additional rows or columns may be considered a control—a control row or control column—for sensor array 40 and allow wireless tag 50 to sense the size of sensor array 40. FIG. 4, as an example, illustrates a control column 48 which is connected to sensor array 40 using resistors R1S, R2S, and R3S. Control column 48 may allow for automatic detection of the size of sensor array 40, which may allow for automatic identification of the size of sensor array 40 when wireless tag 50 is connected to it. Similarly, this control column or control row may be used to detect when wireless tag 50 is connected to a new housing 20. Control column 48 also may be used to detect when a portion of medication compartments 22, such as a column of medication compartments 22, are removed at a perforated line 28.

FIG. 5 is a schematic illustration of an equivalent circuit of sensor array 40 of FIG. 4, in accordance with an embodiment of the present disclosure. Relative to FIGS. 4-5, the operation for scanning resistors 42 on an individual level is described. Specifically, FIG. 5 illustrates the situation where the center row of sensor array 40 of FIG. 4 is driven low and all other rows and columns are driven high, as indicated by the ‘0’ and ‘1’ designations in FIG. 4, and the center column, identified as ‘Sense Col 2,’ is sensed. While the center row and column of sensor array 40 are used in this example, the same process may be repeated for all rows and all columns in sensor array 40. Using this example, within FIG. 5, the goal may be to detect the presence or absence of resistor 42 identified as R22. If resistor 42 is broken, the voltage sensed on column 2 may be the high-level voltage. If resistor 42 is intact, the voltage sensed at column 2 may be the voltage division ratio of (R12 and R32 in parallel) and R22. If the resistors 42 are all of the same value, the measured voltage at Sense Col2 would equal 0.667 times the voltage (0.667*V). If either resistor 42 identified as R12 or R32 is broken, then this voltage is lower. The logic at each location is that, if the voltage is below 0.667V (plus a tolerance margin), then this location can be identified as being broken, which is indicative of the corresponding medication compartment having been opened.

Using the process described relative to FIGS. 4-5, sensing circuit 56 (FIG. 1) may scan sensor array 40 successfully to sense an operation of each of the plurality of resistors 42 individually. It is noted that sensing circuit 56 (FIG. 1) may scan sensor array 40 at various increments, such as only once every minute or several minutes. The scanning process may drive all the columns to a high level (1) except for one column, which may be sensed with an analog/digital converter. All rows may be driven to a high (1) except for one, which will be driven to a low (0).

FIG. 6 is a schematic illustration of sensor array 40 for a housing 20 with a 3×7 array of medication compartment openings 24 in the system for wireless medication compliance sensing 10 of FIG. 1, in accordance with an embodiment of the present disclosure. Sensor array 40 of FIG. 6 may be an example of a printed sensor array 40 on a sheet of material prior to applying it to housing 20, such as is shown in FIG. 1. Sensor array 40 may be printed in a multilayer printing process, wherein conductive traces are first printed, then an insulating ink is printed at the crossover points, a carbon resistor 42 is printed at the crossover points, and finally an overcoat layer is printed to seal sensor array 40. When this sensor array 40 is printed and die-cut, locating holes may be punched to align this layer with housing 20 (FIG. 1) during final assembly. Sensor array 40 may include a connector array 46 at a side of sensor array 40 which may be connectable to wireless tag 50, as is shown in FIG. 6. As a possible extension of the printing process, it may be possible to print sensor array 40 to decode positions or for providing a serial number or other identification of sensor array 40.

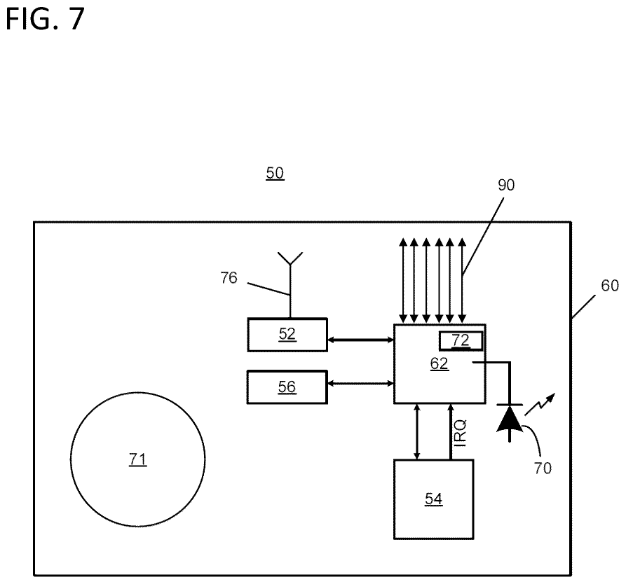

FIG. 7 is a schematic of wireless tag 50 of the system for wireless medication compliance sensing 10, in accordance with an embodiment of the present disclosure. Wireless tag 50 may include a housing 60, which may provide the structure for holding other components of wireless tag 50. Housing 60 may be constructed from a durable material, such as hardened plastic, fiberglass, metal, or another type of material, and may substantially contain wireless transmitter 52, processor 62, and accelerometer 54, along with other components of wireless tag 50. Housing 60 may be scalable and resistant to the elements, such that it is water-resistant, dust-proof, and resistant to other environmental conditions.

Transmitter 52 may include a short-wavelength ultra-high frequency (UHF) radio wave wireless transmitter which transmits a plurality of signals in an ISM band of between 2.4 GHz to 2.485 GHz. Specifically, wireless transmitter 52 may be a 2.4 GHz digital radio transceiver in communication with a printed circuit board (PCB) antenna 76. Processor 62 may be coupled to transmitter 52. Processor 62 may include a microcontroller unit (MCU) that is Bluetooth protocol enabled. One of the benefits of this may be the use of Bluetooth Low-Energy (BLE) protocol, which uses a Bluetooth beacon payload to transmit periodically the scanned sensor array data as well as other data, such as the device ID. This periodic transmission may use less battery power than other wireless protocols, allowing wireless tag 50 to function for longer periods of time. The periodic beacon also may send out battery status and/or signal strength information with the scanned sensor array data to a Bluetooth receiver of an external device. Thus, upon receipt of the beacon, the external device may detect that wireless tag 50 is operational and properly connected to sensor array 40. Other wireless protocols, such as Wi-Fi, also may be used in some instances.

Accelerometer 54 may be in communication with processor 62, wherein accelerometer 54 uses less than 10 μAh of power. Accelerometer 54 may include, for example, a microelectromechanical systems (MEMS) accelerometer in two-way communication with processor 62. Battery 71 may be positioned within housing 60 and provide a quantity of power to processor 62 and accelerometer 54. Housing 60 also may include an indicator 70, a timer 72, and a sensor array input 90 connected to processor 62, which facilitates connection of sensor array 40 (FIG. 1) to wireless tag 50. Indicator 70 may be a light-emitting diode (LED) indicator which is housed at least partially within housing 60 but is visible from a position external of housing 60. Timer 72 may be integrated within processor 62.

The MCU may execute the Bluetooth protocol from stored program code. The MCU may have permanent storage for a quantity of computer programs and permanently may store configuration and operating parameters of the Bluetooth protocol. To save power, the MCU normally may be in a sleep state where it is not running any code. The MCU may be woken up to run code either from an interrupt from one of the devices on the board or by an internal timer. MEMS accelerometer 54 may be configured to detect various events: motion, double-tap, or orientation change. MEMS accelerometer 54 may wake up processor 62 by means of an interrupt request (IRQ) or interrupt signal, and the MCU may send control parameters and read data from accelerometer 54. Thus, upon detection of the event, MEMS accelerometer 54 may generate an interrupt signal to the MCU, which in turn causes the MCU to wake up from a sleep state and process the event.

The MCU also may wake up based on an internal timer 72. An antenna 76 may be included for the MCU to transmit and receive radio frequency (RF) energy. The MCU may utilize power management to go to a low-power sleep state. Wireless tag 50 may not perform a Bluetooth connection protocol to transfer the sensor information, as it is normally transmitting only using the beacon format. Thus, the client receiver does not have to be associated with wireless tag 50 to receive the information.

The use of a single or double tap detected by accelerometer 54 may be used to signal an initial device configuration, may associate wireless tag 50 with an asset by sending special signal code for identification, and may allow a connection between Bluetooth client and host. The orientation of wireless tag 50 when it is tapped may be used to either turn it on or off, and a different orientation may be used to turn it off or on, respectively. When it is turned off, it may no longer be transmitting RF packets. The turn-off function may be disabled when the device is configured. The configuration optionally may be locked and never changed. A secure key code may be stored permanently, and only clients that have the key code may be able to connect and change the operating parameters. The Bluetooth beacon repetition rate may be changed to a higher rate upon a double tap for a period, and a code may be sent as part of the beacon to signal the double tap. The double tap connection to the client may be disabled with a configuration parameter. This may prevent unauthorized changes to the device setup.

When accelerometer 54 generates a motion detection interrupt, motion detection may be enabled or disabled, motion sensitivity and axis of acceleration may be configured, and an indicator 70 LED may flash to show the motion has been detected. The Bluetooth beacon repetition rate may be changed to a higher rate upon motion detection for a period, and a code may be sent as part of the beacon to signal the motion detection. The maximum amount of time in the motion detected state may be configured. This may prevent wireless tag 50 from using up battery 71 when it is in motion for a long period of time, as in truck transport. Minimum motion off time may be provided before re-enabling motion detection, such as, for example, to prevent the motion state being entered every time a truck carrying wireless tag 50 stops at a traffic light. When accelerometer 54 generates an interrupt due to a change in orientation, orientation changes may be configured and enabled, and orientation may change time delay configuration. Wireless tag 50 may include a “panic” button input used to generate an interrupt to the MCU.

The rules and protocols that are used to operate wireless tag 50 may be configured to control the beacon transmission rate. These rules may be based on time and sensor inputs to provide an immediate alert status and then to reduce the beacon repetition rate to lower usage of battery 71. When wireless tag 50 is set to airplane mode of operation, it may not be transmitting beacons in normal operation; it may be waiting for a signal from another device to start transmitting. After the beacons are sent for a programmable period, wireless tag 50 then may go back to a receive-only mode.

Depending on the selected battery 71, wireless tag 50 may operate, for example, for five years or more. To save power, the MCU may be in a sleep mode most of the time. The MCU may wake up from one of several sources: internal timer 72, interrupt from another device in the system, or from a sensor. Internal timer 72 may be used periodically to transmit a signal or to monitor sensors or voltages. One of the possible sources for the external interrupt wake-up may be MEMS accelerometer 54. Internal timer 72 may be used for the MCU to wake up periodically and monitor a sensor array 40 (FIG. 1) connected to sensor array input 90, which may be connected to an analog-to-digital converter input.

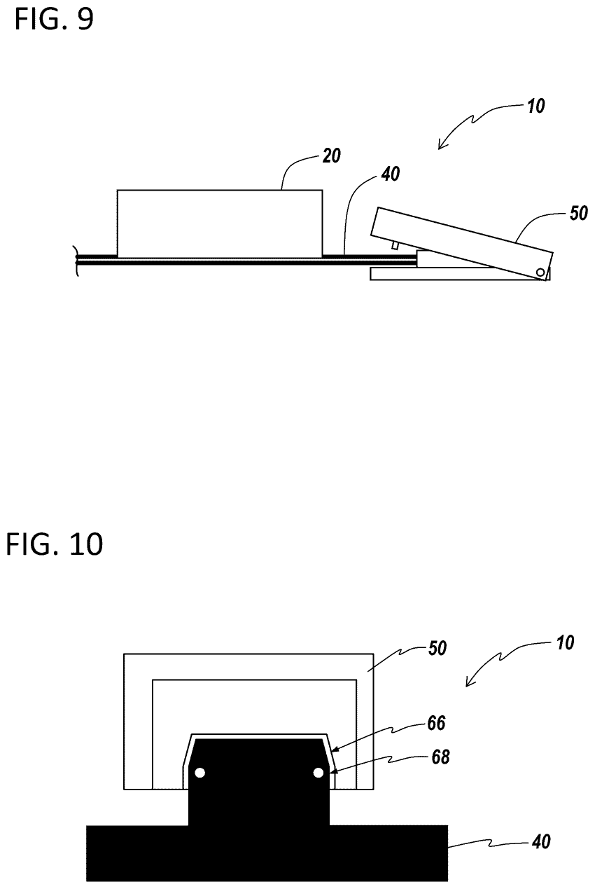

FIG. 8 is a schematic of the system for wireless medication compliance sensing 10, in accordance with an embodiment of the present disclosure. FIG. 8 illustrates a typical system connection to transmit the data from medication compliance system 10 to a computerized device 12, such as a cellphone, a tablet computer, and/or a data bridge. As is shown, a plurality of wireless tags 50 may be used within system 10 in combination with one another and in combination with a computerized device 12. As is shown in FIG. 8, each wireless tag 50 may be secured to a housing 20 of a blister pack. Wireless tag 50 may be secured to housing 20 in any of a variety of ways, such as is discussed relative to FIGS. 8-9. Transmitter 52 of wireless tag 50 may transmit signals 58 to computerized device 12, depicted as a smart phone, in a variety of ways. For example, in one design, signal 58 may be transmitted externally from wireless tag 50 without pre-identification of the computerized device 12 receiving it. In other words, the signal beacon may be transmitted to any available device located within the vicinity, which in turn, can identify signal 58.

Computerized device 12 may include any type of computer, computer system, or other device utilizing a computer processor. For example, computerized device 12 may include a personal computer (PC), a laptop computer, a notebook computer, a computerized smart phone, cellular phone, a PDA, a computerized tablet device, or another device. Commonly, computerized device 12 may be a smartphone, such as an iPhone®, an Android™ phone, or any other cellular phone. Similarly, computerized device 12 may include an interface device, such as a gaming system or a home automation device (e.g., a WINK® device), or any other computerized device capable of receiving a signal 58. Computerized device 12 may include a variety of hardware and software components, including one or more processors, memory units, databases, and/or programs or software applications, all of which are considered within the scope of the present disclosure. For example, computerized device 12 may have a computerized program installed within a memory device therein. The computerized program may be any application software, which may be referred to in the industry as an application or more simply an “app.” Current examples of these apps are commonly referred to by the entity that creates, markets, or sells the app, such as Apps for iPhone® sold at an app store or Google® apps. The app may include software code for performing a single action or multiple, related actions or tasks. The app may be compatible with, or used in conjunction with, any other type of system software, middleware, or program.

System 10 may be enabled with conventional hardware components and software programs as well as specific apps installed within computerized device 12 to receive signal 58 transmitted from wireless tag 50. For example, signal 58 may be received on a wireless receiver, such as a Bluetooth receiver, within computerized device 12 capable of receiving short-wavelength UHF radio waves in an ISM band of between 2.4 GHz and 2.485 GHZ. The functioning of the various components of system 10 and computerized device 12 may utilize a combination of existing software within computerized device 12 for transmitting and receiving wireless signals 58. For example, conventional software may include software associated with the functioning of Bluetooth communication within computerized device 12.

Computerized device 12, through the software operating thereon, may provide a graphical user interface (GUI) 16 or display capable of displaying information about the wireless tag 50. GUI 16 of computerized device 12 may include a listing or indexing of wireless tags 50 that have been detected. Each wireless tag 50 may correspond to an item within the list displayed on GUI 16, and each item displayed may have information indicative of the corresponding system 10. For example, each item displayed may have an identification number of the wireless tag 50 and an indication of activation of wireless tag 50, among other information. The indication of activation of wireless tag 50 may be a color-coded system, whereby wireless tags 50 that are currently activated (i.e., wireless tags 50 that have accelerometers 54 that are experiencing an activation) are identified in one color, whereas inactive wireless tags 50 are identified in a different color. GUI 16 further may include other information about wireless tags 50, including a listing of the total number of wireless tags 50 detected.

Although not required, there are system configurations where wireless tag 50 may be monitored on a server 13 using a data bridge, where server 13 receives information from computerized device 12. In this case, the data is forwarded to server 13 over the data bridge (e.g., standard network lines or wireless channels). The database in server 13 may be viewable using a standard web interface from any computer network, such as from a remotely positioned computer 14 which has a web browser for viewing the data. This data bridge may transfer the short-range signal 58 from wireless tag 50 to the Internet, where the data, including a data packet with ID, may be stored in a server 13 database. The database may be viewed from any Internet-connected device using a web browser when logged in to server 13. At the level of server 13, a timestamp of the receipt of the signal having the scanned data may be logged or recorded. Performing this log at the level of server 13 may allow wireless tags 50 to be free from having to record timing, which may eliminate setup of wireless tag 50 altogether. Wireless tag 50 may transmit a beacon which contains the ID of the package plus additional sensor information. Alternatively, the smartphone or bridge may add a timestamp to the data when it is forwarded to server 13. In one example, the beacon may be transmitted every ten seconds, repeating the same data each time. The smartphone or bridge may determine whether there is a change in the data packet and only send updates to server 13 indicating a change in the number of medication usage.

The data received at the level of server 13 may be reviewed, verified, or otherwise analyzed by various parties, including the medical professional issuing the medication to the patient, to confirm that the medication was taken by the patient or not. If the data is indicative of the medication being used (or not used) in a way different from what was prescribed by the medical professional, system 10 can be configured to send out alerts for non-compliance.

FIG. 9 is a side-view schematic illustration of the connectable wireless tag 50 for use with the system for wireless medication compliance sensing 10, in accordance with an embodiment of the present disclosure. FIG. 10 is a top view schematic illustration of the connectable wireless tag 50 for use with the system for wireless medication compliance sensing 10, in accordance with an embodiment of the present disclosure. Relative to FIGS. 9-10, wireless tag 50 may be attached to sensor array 40 on housing 20 using a variety of mechanical systems. For example, wireless tag 50 may be removably connectable to sensor array 40 with a spring clip or spring-loaded hinged connection which is clipped onto a portion of sensor array 40. When wireless tag 50 mechanically clips onto sensor array 40, electrical contacts on wireless tag 50 may connect electrically with sensor array 40 to facilitate an electrical connection between the two structures. The spring clip, as shown in FIG. 9, may include a wireless tag 50 with an upper portion and a lower portion and hinge therebetween. A spring or similar biasing element may allow the upper and lower portions to close on sensor array 40 and remain biased in the closed position.

There are several other designs which may be used for attaching wireless tag 50 to sensor array 40. For example, a preferred method may be to have wireless tag 50 slide onto sensor array 40 with guides which align contact(s) on wireless tag 50 to the printed contacts on senor array 40. A clamshell-like opening on the side of wireless tag 50 may allow the user to slide it into place without any resistance. Snapping wireless tag 50 closed may lock it onto sensor array 40. A simple snap release may allow wireless tag 50 to be opened and removed from sensor array 40 for reuse.

Wireless tag 50 also may utilize an alignment guide 66 for aligning wireless tag 50 to sensor array 40. Alignment guide 66 may include a guiding structure on wireless tag 50 which aligns with predetermined alignment holes 68 on sensor array 40. To properly align sensor array 40 to wireless tag 50, the user may engage alignment holes 68 with posts of alignment guide 66. This alignment may position conductive traces of sensor array 40 properly with the proper electrical contacts of wireless tag 50.

### II—Example Methodology

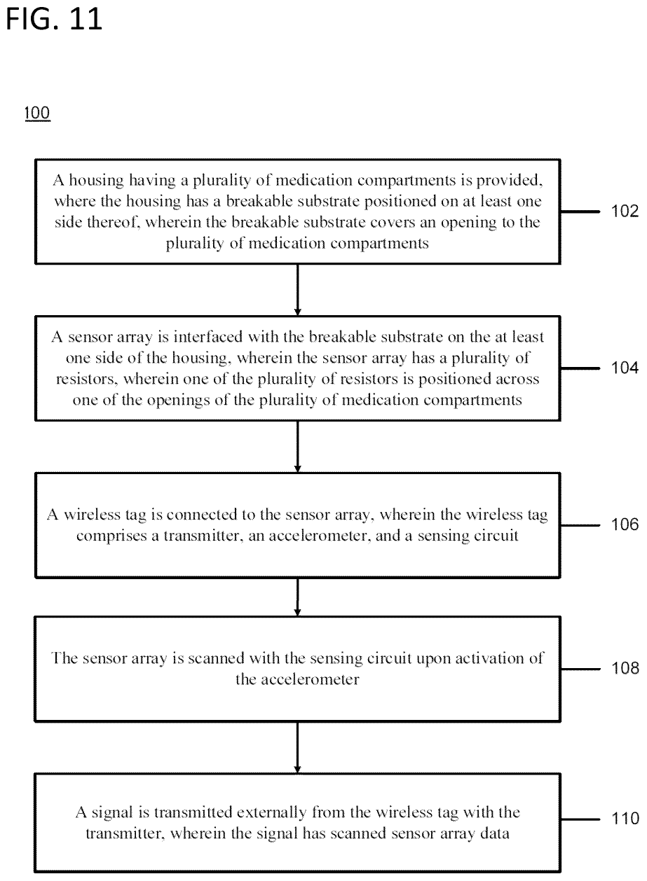

FIG. 11 is a flowchart 100 of a method for wirelessly sensing medication compliance, in accordance with an embodiment of the present disclosure. It should be noted that any process descriptions or blocks in flow charts should be understood as representing modules, segments, portions of code, or steps that include one or more instructions for implementing specific logical functions in the process, and alternate implementations are included within the scope of the present disclosure in which functions may be executed out of order from that shown or discussed, including substantially concurrently or in reverse order, depending on the functionality involved, as would be understood by those reasonably skilled in the art of the present disclosure.

As is shown in FIG. 11, a housing having a plurality of medication compartments may be provided, where the housing may have a breakable substrate positioned on at least one side thereof, wherein the breakable substrate may cover an opening to the plurality of medication compartments (block 102). A sensor array may be interfaced with the breakable substrate on the at least one side of the housing, wherein the sensor array may have a plurality of resistors, wherein one of the plurality of resistors is positioned across one of the openings of the plurality of medication compartments (block 104). A wireless tag may be connected to the sensor array, wherein the wireless tag may include a transmitter, an accelerometer, and a sensing circuit (block 106). The sensor array may be scanned with the sensing circuit upon activation of the accelerometer (block 108). A signal may be transmitted externally from the wireless tag with the transmitter, wherein the signal may have scanned sensor array data (block 110).

The method may include any number of additional steps, processes, or functions, including all disclosed within this disclosure. For example, the signal may be externally transmitted from the housing using the wireless transmitter transmitting the signal using short-wavelength UHF radio waves in an ISM band of between 2.4 GHz and 2.485 GHz. A quantity of power may be provided to at least the processor and the accelerometer, wherein the accelerometer may use less than 10 μAh of the quantity of power. Scanning the sensor array with the sensing circuit upon activation of the accelerometer may include sensing a break in the breakable substrate and one of the plurality of resistors over the opening to one of the plurality of medication compartments with a wireless tag connectable to the sensor array. Scanning also may include using at least one of a control row and a control column to sense a size of the sensor array. Scanning the sensor array with the sensing circuit upon activation of the accelerometer also may include scanning the sensor array to sense an operation of each of the plurality of resistors individually.

After the signal with the scanned sensor array data is transmitted externally from the wireless tag with the transmitter, the method optionally may include a number of other steps. For example, the scanned sensor array data may be forwarded from the data bridge to a server for processing or analysis of the scanned sensor array data. In one example, a medication compliance application may be used to analyze the scanned sensor array data relative to a predefined medication regimen to determine whether the scanned sensor array data indicates compliance or non-compliance between the patient's usage of the medication and the specified regimen. The system may communicate various notifications to the patient, to the prescribing medical professional, or to another party, such as a caregiver, to indicate compliance with the medication regimen or to indicate that further action is needed. The system also can be used to send reminders to patients using automated electronic communications (e.g., such as text messages, e-mails, phone calls, etc.).

### III—Additional Example Device, System Architecture, and Operation

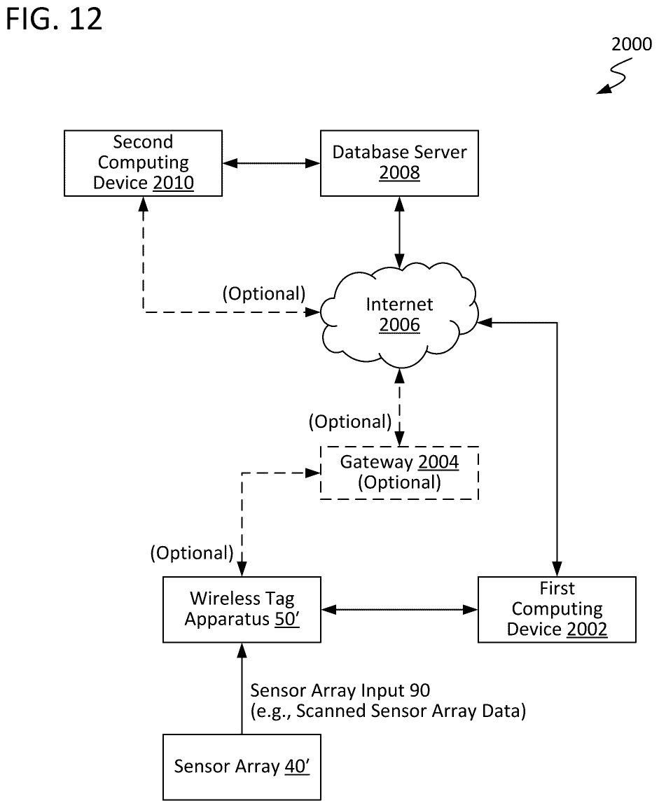

FIG. 12 illustrates a medication compliance monitoring system 2000 configured in accordance with an embodiment of the present disclosure. As can be seen, system 2000 may include a wireless tag apparatus 50′ configured, in accordance with some embodiments, to be communicatively coupled with a sensor array 40′ (associated with a medication dispenser external to apparatus 50′) to receive sensor array input 90 (e.g., scanned sensor array data) therefrom. As can be seen further, system 2000 may involve in its operation one or more of a first computing device 2002, a gateway 2004, the internet 2006, a database server 2008, and a second computing device 2010. Each of these various elements is discussed in turn below. More generally, FIG. 12 illustrates communicative coupling of the various constituent elements of system 2000 and the overall flow of data within system 2000, in accordance with some embodiments.

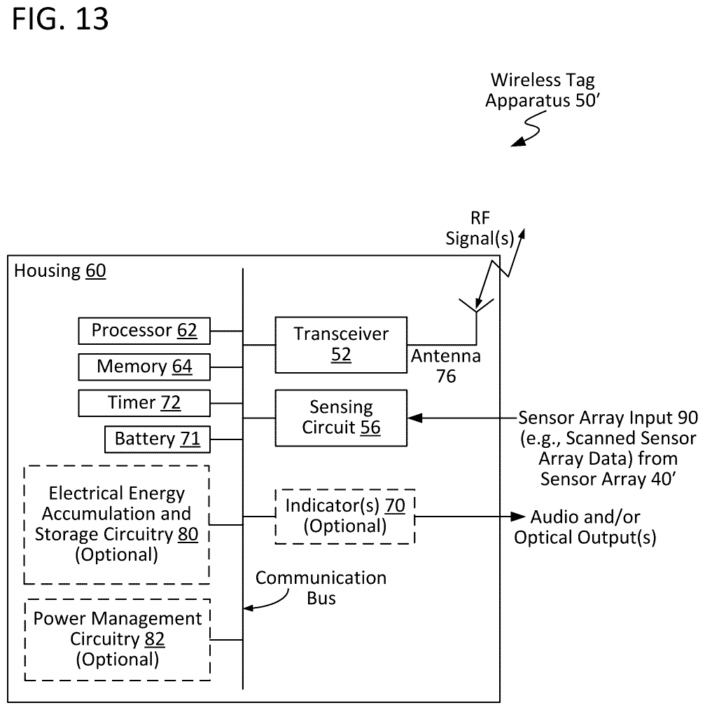

As noted above, system 2000 may involve use of a wireless tag apparatus 50′. FIG. 13 is a block diagram of a wireless tag apparatus 50′ configured in accordance with an embodiment of the present disclosure. As will be apparent in light of this disclosure, apparatus 50′ may include one or more components configured the same as (or similar to) correspondingly numbered components described herein with respect to apparatus 50, in accordance with some embodiments. Additional details regarding embodiments of apparatus 50′ are provided below.

In accordance with some embodiments, apparatus 50′ may be configured to detect the status of sensor array 40′ (discussed below) in part or in whole and determine when contents of at least one medication compartment of the medication dispenser have been expelled and to transmit one or more RF signals including data pertaining to such information. To such ends, apparatus 50′ may be configured, in some embodiments, to be connected to sensor array 40′ in the same (or similar) way as apparatus 50 (described above) is connectable to sensor array 40.

In accordance with some embodiments, apparatus 50′ may include memory 64, which may be implemented with any one or combination of types of non-volatile memory and may be of any type and size, as desired for a given target application or end-use. In some cases, memory 64 may be configured for use in storing data, on a temporary or permanent basis, whether that data is native to apparatus 50′ or received from another source. In some instances, memory 64 may be configured to store data obtained from scanning of sensor array 40′ via sensing circuit 56. In some instances, memory 64 may be configured for use as processor workspace for processor 62.

In accordance with some embodiments, memory 64 may include, for example, a computer-readable medium that, when executed by a processor (e.g., such as processor 62), carries out any one or more of the functions described herein, in part or in whole. The computer-readable medium may be, for example, a hard drive, a compact disk, a memory stick, a server, or any other suitable non-transitory computer or computing device memory that includes executable instructions, or a plurality or combination of such memories. Other embodiments can be implemented, for instance, with gate-level logic or an application-specific integrated circuit (ASIC) or chip set, or other such purpose-built logic. Some embodiments can be implemented with a microcontroller having input/output (I/O) capability (e.g., inputs for receiving user inputs; outputs for directing other components) and one or more embedded routines for carrying out device functionality. In a more general sense, memory 64 may be implemented in hardware, software, firmware, or a combination thereof, as desired for a given target application or end-use. Other suitable configurations for memory 64 will depend on a given application and will be apparent in light of this disclosure.

In accordance with some embodiments, apparatus 50′ may include a processor 62, which may be configured to communicate with any one (or combination) of the other various components of apparatus 50′ via a communication bus or other suitable interconnect. Processor 62 may be, for example, a central processing unit (CPU), a microcontroller unit (MCU), or any other suitable processing element, as will be apparent in light of this disclosure. In performing a given operation associated with apparatus 50′, processor 62 may be configured to access data, such as scanned sensor array data, stored at memory 64 or otherwise accessible to apparatus 50′. When sensing circuit 56 receives scanned sensor array data from a sensor array 40′ operatively coupled with apparatus 50′, processor 62 may be alerted and may process the data so received, in accordance with some embodiments. As will be appreciated in light of this disclosure, processor 62, as employed here in the context of apparatus 50′, may be configured as variously described above with respect to apparatus 50, in accordance with some embodiments. Other suitable configurations for processor 62 will depend on a given application and will be apparent in light of this disclosure.

In accordance with some embodiments, apparatus 50′ may include a transceiver 52, which may be provided with both receiving and transmitting capabilities. In accordance with some embodiments, transceiver 52 may be a radio-wave wireless communication device configured to receive and/or transmit signals of a frequency in any one (or combination) of wireless communication bands, such as, for example, Bluetooth (e.g., 2.4 GHZ), LoRaWAN (e.g., 900 MHZ), and Wi-Fi (e.g., 2.4 GHz, 5.0 GHZ), to name a few. As a wireless communication device, transceiver 52 may include an antenna 76 configured to receive and/or transmit one or more signals. To that end, antenna 76 may be, for example, a PCB antenna configured as typically done or any other suitable antenna, as will be apparent in light of this disclosure. Other suitable configurations for transceiver 52 and its antenna 76 will depend on a given application and will be apparent in light of this disclosure.

In accordance with some embodiments, apparatus 50′ may include a timer 72 configured to count a given time interval. As will be appreciated in light of this disclosure, the duration of the time interval may be customized, as desired for a given target application or end-use. For example, in some cases, the time interval may be on the order of minutes or seconds. In some instances, the time interval may range from one minute to twenty-four hours. In some instances, the time interval may range from about 5 minutes to 1 hour. In any case, the selected time interval may be one which allows for a sufficient level of accuracy for determining whether individual medication dosage(s) have been missed, as well as for determining whether a given device or system error or problem might have occurred. In short, the time interval may be tailored to provide a given target level of detail necessary for monitoring (and acting, if needed) in a timely manner.

In accordance with some embodiments, at the completion of a given time interval, timer 72 may output a signal to processor 62 indicating that the time interval has expired. In response to completion of the time interval, processor 62 may instruct transceiver 52 to transmit RF signal(s) including data pertaining to (A) scanned sensor array data obtained by the scanning of sensor array 40′ by sensing circuit 56 of apparatus 50′ and/or (B) data relating to apparatus 50′ more generally. Other suitable configurations for timer 72 will depend on a given target application or end-use and will be apparent in light of this disclosure.

In accordance with some embodiments, apparatus 50′ optionally may include one or more indicators 70, which may include any one (or combination) of audio and optical output devices. For instance, indicator(s) 70 may include an audio output device, which may be a speaker, beeper, or any other device capable of emitting sound of a given frequency, optionally with a given emission period or pattern. Additionally (or alternatively), indicator(s) 70 may include an optical output device, which may be a light source, such as a light-emitting diode (LED) or other solid-state light engine, or any other device capable of emitting light of a given wavelength, optionally with a given emission period or pattern.

In accordance with some embodiments, a given indicator 70 may be configured to output audio output signal(s) and/or optical output signal(s) indicative of a given condition with respect to the operation of apparatus 50′ (or system 2000 more generally). For instance, in some cases, indicator 70 may emit a sound and/or light indicative of a low power level of battery 71. In some cases, indicator 70 may emit a sound and/or light indicative of removal of apparatus 50′ from an associated sensor array 40′. In some cases, indicator 70 may emit a sound and/or light indicative of a malfunction of sensor array 40′ or apparatus 50′. Other suitable configurations and uses for optional indicator(s) 70 including audio and/or optical output devices will depend on a given application and will be apparent in light of this disclosure.

In accordance with some embodiments, apparatus 50′ optionally may include electrical energy production and storage circuitry 80 configured to produce electrical energy from any one or combination of energy sources (e.g., movement, solar, thermal, etc.) and provide such electrical energy for immediate use or storage, as desired, by apparatus 50′. In some cases, circuitry 80 may include one or more devices configured to convert the energy of movement into electricity. In some cases, circuitry 80 may include one or more photovoltaic devices (e.g., solar power modules or elements) configured to convert the energy of sunlight (or other light) into electricity. Circuitry 80 may include any one (or combination) of electrical energy storage elements, including, for example, batteries, capacitors, or any other device capable of storing electrical energy on a temporary, semi-permanent, or permanent basis. Other suitable configurations and capabilities for electrical energy production and storage circuitry 80 will depend on a given target application or end-use and will be apparent in light of this disclosure.

In accordance with some embodiments, apparatus 50′ optionally may include power management circuitry 82 configured to manage energy consumption for apparatus 50′. To that end, circuitry 82 may be configured, in instances, to switch between drawing energy from battery 71 or drawing energy from electrical energy production and storage circuitry 80. In this manner, circuitry 82 may be configured to assist apparatus 50′ in optimizing its consumption of electrical energy from a given power source. In accordance with some embodiments, while harvesting radiant energy (e.g., RF energy), power management circuitry 82 may have apparatus 50′ rely on drawing power from battery 71 until a given charge point for electrical energy production and storage circuitry 80 is reached. In accordance with some embodiments, power management circuitry 82 may utilize harvested solar energy while its output remains constant above a certain milliwatt-hour value (e.g., when sufficient sunlight is present) but then change to battery 71 when the output drops below that value (e.g., when insufficient sunlight is present). Other suitable configurations and capabilities for power management circuitry 82 will depend on a given target application or end-use and will be apparent in light of this disclosure.

It should be noted that apparatus 50′ is not intended to be limited only to the example configuration described above and illustrated in FIG. 13, as numerous other configurations and variations will be apparent in light of this disclosure. For instance, in some other embodiments, any (or all) of memory 64, processor 62, timer 72, and transceiver 52 may be provided as a single device having the capabilities of each attendant component.

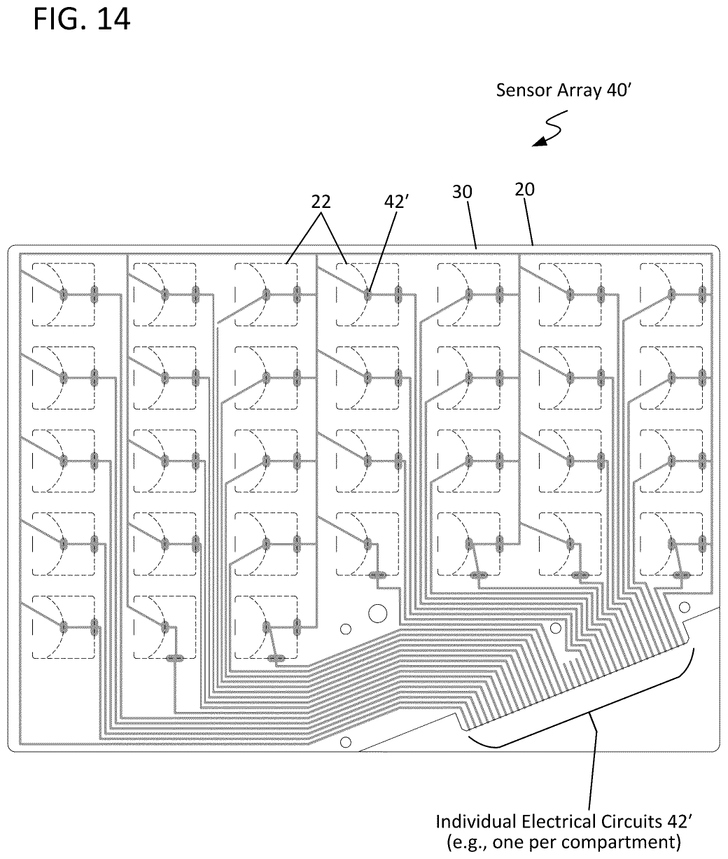

Also, as noted above, system 2000 may involve use of a sensor array 40′. FIG. 14 is a diagram of a sensor array 40′ configured in accordance with an embodiment of the present disclosure. As will be apparent in light of this disclosure, sensor array 40′ may include one or more components configured the same as (or similar to) correspondingly numbered components described herein with respect to sensor array 40, in accordance with some embodiments. Additional details regarding embodiments of sensor array 40′ are provided below.

As can be seen from FIG. 14, sensor array 40′ may include a plurality of electrical circuits 42′ and, at least in some such instances, each individual electrical circuit 42′ may be associated with a single medication compartment 22. More specifically, a given electrical circuit 42′ may be formed such that a portion thereof is disposed over at least one medication compartment 22, in accordance with some embodiments. In at least some cases, a given electrical circuit 42′ of sensor array 40′ may be formed, in part or in whole, via an electrically conductive ink.

In accordance with some embodiments, when the contents of a given medication compartment 22 are expelled, the associated electrical circuit 42′ may be broken locally, and thus apparatus 50′ may determine with confidence precisely which medication compartment 22 has been affected. Conversely, if the contents of a given medication compartment 22 have not been expelled but should have been, the associated electrical circuit 42′ may remain intact (i.e., unbroken), and thus apparatus 50′ may determine with confidence precisely which medication compartment 22 was not accessed (and thus which dosage was missed).

As will be appreciated in light of this disclosure, a given electrical circuit 42′ (of sensor array 40′) may differ from a given resistor 42 (of sensor array 40, described above) at least in that electrical circuit 42′ is not limited to being a resistive element like resistor 42. In this sense, sensor array 40′ may be configured to operate such that the integrity of a given individual electrical circuit 42′ may be determined based on whether said circuit is closed (i.e., unbroken) or open (i.e., broken), signifying when a given medication compartment 22 has been accessed. In a different way, sensor array 40 may involve determining changes in electrical resistance levels based on having one or more resistors 42 being taken out of the overall circuit when a given medication compartment 22 has been accessed.

Returning to FIG. 12, system 2000 further may involve use of a first computing device 2002, mobile or otherwise, in accordance with some embodiments. First computing device 2002 may be any one (or combination) of a laptop/notebook computer, a sub-notebook computer, a tablet computer, a desktop computer, a mobile phone, a smartphone, a personal digital assistant (PDA), a cellular handset, a television set, a computer monitor, a media streaming device, and a video conferencing system, among other options. First computing device 2002 may be configured for communication with apparatus 50′ utilizing wired communication via Universal Serial Bus (USB), Ethernet, FireWire, or other wired communicating interfacing, wireless communication via Wi-Fi, Bluetooth, or other wireless communication interfacing, or a combination of any thereof. In accordance with some embodiments, first computing device 2002 may host a browser or other software application configured to facilitate review of information pertinent to sensor array 40′, apparatus 50′, and/or any part or the totality of system 2000 and its various constituent elements.

In accordance with some embodiments, first computing device 2002 may be configured for monitoring and/or analyzing the operation of one or more of sensor array 40′ and apparatus 50′. In some cases, first computing device 2002 may be a dedicated reader device configured specifically to such ends, whereas in some other cases, first computing device 2002 may be a general computing device configured for use to such ends, optionally hosting an application to facilitate its use in monitoring and/or analyzing sensor array 40′ and/or apparatus 50′. First computing device 2002 may be configured, in accordance with some embodiments, to access database server 2008 (discussed below) to display current and/or previous data pertaining to sensor array 40′ and apparatus 50′, including scanned sensor array data obtained by apparatus 50′ from scanning sensor array 40′.

In accordance with some embodiments, first computing device 2002 may be configured to receive scanned sensor array data from apparatus 50′ upon receipt of a successfully secure pairing handshake. As will be appreciated in light of this disclosure, this handshake may include, for example, a request for a Bluetooth LE (or other suitable) connection to apparatus 50′. Once the connection is confirmed, apparatus 50′ may request an encrypted password. If the encrypted password is correct, apparatus 50′ may issue a standard Bluetooth (or other) pairing request, which the originating first computing device 2002 can accept, and then two-way inspection and/or update to apparatus 50′ can continue. Other suitable configurations for first computing device 2002 will depend on a given target application or end-use and will be apparent in light of this disclosure.

In accordance with some embodiments, system 2000 optionally may involve use of a gateway 2004 configured to receive data gathered by apparatus 50′ (e.g., scanned sensor array data) and transmit that data to a database server 2008 (discussed below) via the internet 2006. To such ends, gateway 2004 may be configured to utilize any one or combination of suitable communication protocols, wired or wireless, such as, for example, Ethernet, Bluetooth, Wi-Fi, and cellular, among others. In accordance with some embodiments, gateway 2004 may be any one (or combination) of fixed Bluetooth-to-Wi-Fi, cellular-to-Wi-Fi, or cellular-to-Bluetooth bridge/hub devices. Gateway 2004 may be used to read signal(s) from apparatus 50′ within range and to forward information over a network interface to internet 2006 and database server 2008. In some instances, apparatus 50′ may transmit signal(s) including, for example, data obtained from scanning of sensor array 40′ and/or data pertaining to the operation of apparatus 50′ (e.g., such as the current power level of battery 71 or the amount of energy available at electrical energy production and storage circuitry 80), and gateway 2004 may be configured to receive such signal(s) and relay data obtained therefrom to database server 2008. In this manner, a mechanism may be provided by which the status of sensor array 40′ and/or apparatus 50′, in part or in whole, may be determined, monitored, and analyzed. Other suitable configurations for gateway 2004 will depend on a given application and will be apparent in light of this disclosure.

In accordance with some embodiments, system 2000 may involve use of a database server 2008, which may be accessible through the internet 2006 and, in some cases, may be cloud-based, in part or in whole. As a means of data storage, database server 2008 may be configured to store information saved thereat, for instance, by apparatus 50′, first computing device 2002, and/or second computing device 2010 (discussed below). In accordance with some embodiments, database server 2008 may store information about sensor array 40′ (e.g., scanned sensor array data) and/or information about apparatus 50′, which may be retrieved by first computing device 2002 and/or second computing device 2010, for example. In accordance with some embodiments, database server 2008 may be configured to verify that the various constituent elements of system 2000 are working properly. For instance, database server 2008 may be used, in accordance with some embodiments, to monitor the operating status of sensor array 40′. Additionally (or alternatively), database server 2008 may be used, in accordance with some embodiments, to monitor the operating status of apparatus 50′. In a more general sense, database server 2008 may allow for a given desired degree of inter-networking of the components of system 2000 and other elements as part of the internet of things (IoT), in accordance with some embodiments. Other suitable configurations for database server 2008 will depend on a given application and will be apparent in light of this disclosure.

In accordance with some embodiments, system 2000 further may involve use of a second computing device 2010, mobile or otherwise. As will be appreciated in light of this disclosure, the description provided above with respect to the configurations and capabilities of first computing device 2002 also may apply here, in part or in whole, with respect to second computing device 2010, in accordance with some embodiments. In some cases, second computing device 2010 and first computing device 2002 may be the same device.

In accordance with some embodiments, second computing device 2010 may be configured for monitoring and/or analyzing data retrieved from the internet 2006 and/or database server 2008 (e.g., scanned sensor array data obtained from sensor array 40′ through apparatus 50′). In some cases, second computing device 2010 may be a dedicated reader device configured specifically to such ends, whereas in some other cases, second computing device 2010 may be a general computing device configured for use to such ends, optionally hosting an application to facilitate its use in monitoring and/or analyzing the internet 2006 and/or database server 2008. Second computing device 2010 may be configured, in accordance with some embodiments, to access database server 2008 to display current and/or previous data pertaining to sensor array 40′ and apparatus 50′, including scanned sensor array data obtained by apparatus 50′ from scanning sensor array 40′.

In accordance with some embodiments, second computing device 2010 may be configured to present medication compliance monitoring data to a user or other intended audience. To that end, second computing device 2010 may have installed therein (or otherwise have access to) one or more suitable software applications, which may be device-specific or cloud-based in some instances. Other suitable configurations for second computing device 2010 will depend on a given target application or end-use and will be apparent in light of this disclosure.

### IV—Additional Example Methodology

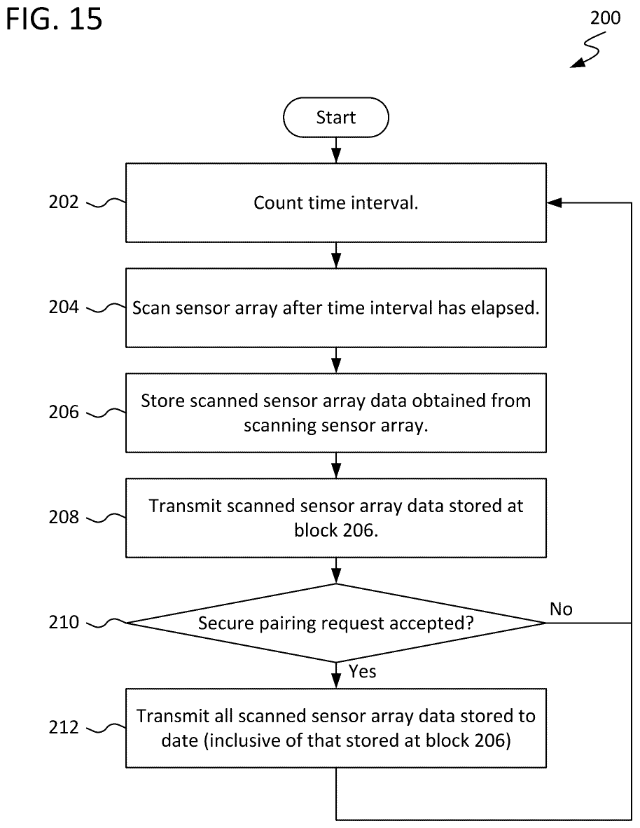

FIG. 15 is a flow diagram illustrating a method 200 of medication compliance monitoring in accordance with an embodiment of the present disclosure. As will be appreciated in light of this disclosure, method 200 may be performed, in part or in whole, via apparatus 50′ in the context of a system 2000, in accordance with some embodiments. It should be noted, however, that method 200 is not intended to be limited only to apparatus 50′ and system 2000 and the various respective constituent elements thereof, as in a more general sense, and in accordance with some embodiments, method 200 may be performed by other suitable structures and devices, as will be apparent in light of this disclosure.

As can be seen, method 200 may begin as in block 202 with counting a time interval. In accordance with some embodiments, counting of the time interval may be performed via timer 72 of apparatus 50′, as described herein.

Method 200 may continue as in block 204 with scanning a sensor array after the time interval has elapsed. In accordance with some embodiments, scanning may be performed via sensing circuit 56 of apparatus 50′, and instruction to perform scanning may be provided via processor 62 of apparatus 50′, as described herein.

Method 200 may continue as in block 206 with storing scanned sensor array data obtained from scanning the sensor array. In accordance with some embodiments, the scanned sensor array data be provided as part of sensor array input 90 received by apparatus 50′, and storage of the scanned sensor array data may be provided via memory 64 of apparatus 50′, as described herein.

Method 200 may continue as in block 208 with transmitting the scanned sensor array data stored at block 206. In accordance with some embodiments, transmission of the scanned sensor array data may be provided by transceiver 52 (with attendant antenna 76), and instruction to perform transmission may be provided via processor 62 of apparatus 50′, as described herein.

Method 200 may continue as in block 210 with determining whether a secure pairing request is accepted. In accordance with some embodiments, determination of whether a secure pairing request is to be accepted may be performed by processor 62 of apparatus 50′, and the secure pairing request may be provided by at least one of a first computing device 2002 and a gateway 2004 (if optionally present), such that the secure pairing event (e.g., secure handshake) may occur between apparatus 50′ and the at least one of the first computing device 2002 and optional gateway 2004.

If a secure pairing request has not been accepted at block 210, method 200 may return to block 202 (described above), and a new time interval may be counted. In accordance with some embodiments, counting of the new time interval may be performed via timer 72 of apparatus 50′, as described herein.

If, however, a secure pairing request has been accepted at block 210, method 200 may continue as in block 212 with transmitting all scanned sensor array data stored to date (inclusive of the scanned sensor array data stored at block 206). In accordance with some embodiments, the scanned sensor array data stored via memory 64 of apparatus 50′ may be retrieved by processor 62 of apparatus 50′, transmission of the scanned sensor array data may be provided by transceiver 52 (with attendant antenna 76), and instruction to perform transmission may be provided via processor 62 of apparatus 50′, as described herein.

After transmission at block 212, method 200 may return to block 202 (described above), and a new time interval may be counted. Again, counting of the time interval may be performed via timer 72 of apparatus 50′, as described herein, in accordance with some embodiments.

It should be emphasized that the above-described embodiments of the present disclosure, particularly, any “preferred” embodiments, are merely possible examples of implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure and the present disclosure and protected by the following claims.

What is claimed is:

1. A wireless tag apparatus comprising:
a timer configured to count a designated time interval;
a sensing circuit configured to scan, after the designated time interval has elapsed, a sensor array associated with a medication dispenser external to the wireless tag apparatus to detect a change to an electrical circuitry of the sensor array corresponding to expulsion of contents of at least one medication compartment of the medication dispenser;
memory configured to store data pertaining to whether the electrical circuitry of the sensor array has so changed; and
a wireless communication module configured to transmit, after the designated time interval has elapsed, a signal including the data pertaining to whether the electrical circuitry of the sensor array has so changed;
wherein the wireless tag apparatus is configured to transmit, in the signal, scanned sensor array data when a secure pairing request from an external computing device is accepted by the wireless tag apparatus.

2. The apparatus of claim 1, wherein the change to the electrical circuitry comprises a breakage of the electrical circuitry.

3. The apparatus of claim 2, wherein the breakage of the electrical circuitry corresponds with electrical circuitry disposed over the at least one medication compartment.

4. The apparatus of claim 1, wherein the medication dispenser comprises a blister pack.

5. The apparatus of claim 1, wherein:
the sensing circuit is further configured to scan the sensor array to determine whether each portion of the sensor array is operational; and
the signal further includes data pertaining to whether each portion of the sensor array is operational.

6. The apparatus of claim 1, wherein the sensing circuit is further configured to detect when the wireless tag apparatus is operatively coupled with the sensor array.

7. The apparatus of claim 1, wherein the wireless communication module comprises a transceiver.

8. The apparatus of claim 1, wherein the signal is a radio frequency (RF) signal.

9. The apparatus of claim 1, wherein the wireless tag apparatus is configured to transmit, in the signal, all scanned sensor array data stored to date when the secure pairing request from the external computing device is accepted by the wireless tag apparatus.

10. The apparatus of claim 1, wherein:
the wireless tag apparatus further comprises a processing element; and
the timer is native to the processing element.

11. The apparatus of claim 1, wherein the memory comprises non-volatile memory.

12. A method of medication compliance monitoring, the method comprising:
counting a designated time interval;
scanning, after the designated time interval has elapsed, a sensor array associated with a medication dispenser to detect a change to an electrical circuitry of the sensor array corresponding to expulsion of contents of at least one medication compartment of the medication dispenser;
storing data pertaining to whether the electrical circuitry of the sensor array has so changed; and
transmitting, after the designated time interval has elapsed, a signal including the data pertaining to whether the electrical circuitry of the sensor array has so changed.

13. The method of claim 12, wherein the change to the electrical circuitry comprises a breakage of the electrical circuitry.

14. The method of claim 13, wherein the breakage of the electrical circuitry corresponds with electrical circuitry disposed over the at least one medication compartment.

15. The method of claim 12, wherein the medication dispenser comprises a blister pack.

16. The method of claim 12, wherein transmitting the signal is performed by a wireless communication module of a wireless tag apparatus configured to perform at least a portion of the method.

17. The method of claim 16, wherein the wireless communication module comprises a transceiver.

18. The method of claim 12, wherein the signal is a radio frequency (RF) signal.

19. The method of claim 12, further comprising:
transmitting, in the first signal, scanned sensor array data when a secure pairing request from an external computing device is accepted.

20. The method of claim 12, further comprising:
transmitting, in the first signal, all scanned sensor array data stored to date when a secure pairing request from an external computing device is accepted.

21. The method of claim 12, wherein counting the designated time interval is performed by a timer of a wireless tag apparatus configured to perform at least a portion of the method.

22. The method of claim 21, wherein the timer is native to a processing element of the wireless tag apparatus.

23. The method of claim 12, wherein storing data pertaining to whether the electrical circuitry of the sensor array has so changed is performed by a memory of a wireless tag apparatus configured to perform at least a portion of the method.

24. The method of claim 23, wherein the memory comprises non-volatile memory.
